(19) United States
(12) Patent Application Publication
(10) Pub. No.: US 2026/0286285 A1
(43) Pub. Date: Sep. 24, 2026

(54) NOVEL PROCESS FOR PRODUCING LACTOBACILLUS RHAMNOSUS

(71) Applicant: DSM IP ASSETS B.V., Maastricht (NL)

(72) Inventors: Henderikus Bernardus Albertus WEGKAMP, Echt (NL); Paul ZUUR, Echt (NL)

(21) Appl. No.: 19/490,039

(22) PCT Filed: Jun. 10, 2024
(86) PCT No.: PCT/EP2024/065888

§ 371 Date: Dec. 4, 2025

(30) Foreign Application Priority Data
Jun. 8, 2023 (EP) EP23178307.7
Jun. 8, 2023 (EP) EP23178308.5
Jun. 8, 2023 (EP) EP23178309.3
Jun. 9, 2023 (EP) EP23178554.4
Jun. 9, 2023 (EP) EP23178555.1
Jun. 9, 2023 (EP) EP23178556.9

(51) Int. Cl. C12N 1/20; A23L 2/52; A23L 33/135; C12N 1/02; C12N 1/04; C12R 1/225

(52) U.S. Cl. CPC C12N 1/20; A23L 2/52; A23L 33/135; C12N 1/02; C12N 1/04; C12R 2001/225

(57) ABSTRACT

A process for the production of a Lactobacillus rhamnosus, wherein the process comprises the steps of:
a) fermenting the Lactobacillus rhamnosus in a lactose-free, preferably milk-free, medium in a fermentor, and removing a fermentation broth from the fermentor, wherein the fermentation is carried out in the presence of acetate or a acetate salt;
b) applying shear to the fermentation broth and optionally concentrating the fermentation broth.

Compositions so obtained and use of such compositions.

### FIELD OF THE INVENTION

The invention relates to a novel process for producing Lactobacillus rhamnosus and for producing a composition comprising Lactobacillus rhamnosus. In addition, the invention relates to such a composition comprising Lactobacillus rhamnosus, the use of such a composition in a food or beverage and the use of such a composition for medical purposes.

### BACKGROUND OF THE INVENTION

Bacterial compositions having probiotic activity are becoming increasingly popular as a part of human and animal diet due to their beneficial health effects. These health benefits, in addition to supporting intestinal health and function, include repopulating the gut after antibiotic therapy, offsetting lactose intolerance, supporting the immune system and reducing cholesterol. Lactic acid bacteria, primarily from the Lactobacillus and Bifidobacterium genera, that can help improve or maintain intestinal health and function are often termed probiotic bacteria (also referred to herein as probiotics).

Lactobacillus rhamnosus, and especially Lactobacillus rhamnosus GG is one of the most popular probiotic bacteria.

In their article titled “Functional Analysis of Lactobacillus rhamnosus GG Pili in Relation to Adhesion and Immunomodulatory Interactions with Intestinal Epithelial Cells”, published in Applied and Environmental Microbiology, Volume 78, Number 1, January 2012, pages 185-193, Lebeer et al. analyse the function of pili for Lactobacillus rhamnosus GG. They indicate that the SpaCBA pilus of Lactobacillus rhamnosus GG was key for efficient adherence to the intestinal epithelial cell line and for biofilm formation.

Pili (also referred to as fimbria) are small hair-like fibrous proteins that are present on the surface area of many bacteria. In order to improve the bioavailability of the pili, it would be an advantage to increase the surface area of the Lactobacillus rhamnosus GG bacterium per gram of product, for example by having more bacterial particles per gram and/or by having smaller bacterial particles.

In addition, probiotics are preferably sold based on their count of colony forming units (CFU) per gram. The count of colony forming units (CFU) per gram is looked at by customers as a measure for the viability of the probiotic. Manufacturers of probiotics therefore consider it an advantage to have a high count of CFU/g in their product.

Thus there remains a need in the art for a process to produce Lactobacillus rhamnosus with a high bioavailability of pili and/or a high bacterial surface area and/or a small bacterial particle size and/or a high count of CFU/gram and/or a high viable cell count.

### SUMMARY OF THE INVENTION

Novel processes for producing Lactobacillus rhamnosus and for producing a composition comprising Lactobacillus rhamnosus have now been found. With these processes the bioavailability of pili and/or the bacterial particle size and/or the count of CFU/gram and/or the viable cell count of the Lactobacillus rhamnosus can be improved.

Accordingly, in a first aspect, the invention provides a process for the production of a Lactobacillus rhamnosus, wherein the process comprises the steps of:
(a) fermenting the Lactobacillus rhamnosus in a lactose-free, preferably milk-free, medium in a fermentor, and removing a fermentation broth from the fermentor, wherein the fermentation is carried out in the presence of acetate or a acetate salt;
(b) applying shear to the fermentation broth and optionally concentrating the fermentation broth.

Further, in a second aspect, the invention provides a process for the production of a composition comprising a Lactobacillus rhamnosus, wherein the process comprises the steps of:
(a) fermenting the Lactobacillus rhamnosus in a lactose-free, preferably milk-free, medium in a fermentor, and removing a fermentation broth from the fermentor, wherein the fermentation is carried out in the presence of acetate or a acetate salt;
(b) applying shear to the fermentation broth and optionally concentrating the fermentation broth.

In a third aspect, the invention provides Lactobacillus rhamnosus cells, respectively Lactobacillus rhamnosus bacterial particles, or a composition comprising Lactobacillus rhamnosus cells, respectively Lactobacillus rhamnosus bacterial particles, obtained or obtainable by any one of the above processes.

In a fourth aspect, the invention provides Lactobacillus rhamnosus cells, respectively Lactobacillus rhamnosus bacterial particles, wherein equal to or more than 90% w/w, preferably equal to or more than 95% w/w, more preferably equal to or more than 99% w/w, yet more preferably equal to or more than 99.5% w/w, still more preferably equal to or more than 99.9% w/w and most preferably still more preferably equal to or more than 100.0% w/w of the bacterial particles have a particle size, suitably as determined by laser diffraction particle size analysis, diameter of equal to or less than 8.0 micrometer (μm), preferably equal to or less than 7.5 micrometer, more preferably equal to or less than 7.0 micrometer, yet more preferably equal to or less than 6.5 micrometer and most preferably equal to or less than 6.0 micrometer preferably equal or less than 5.5, 5.0, 4.5, 4.0, 3.5, 3.0, 2.5, or 2.0 micrometer and the cells preferably have a cell viability of equal to or more than 2.00·10¹⁰ CFU/gram, more preferably a cell viability of equal to or more than 2.10·10¹⁰ CFU/gram, yet more preferably a cell viability of equal to or more than 1.50·10¹¹ CFU/gram and most preferably a cell viability of equal to or more than 2.00·10¹¹ CFU/gram.

In a fifth aspect, the invention provides a composition comprising Lactobacillus rhamnosus cells, respectively Lactobacillus rhamnosus bacterial particles, wherein equal to or more than 90% w/w, preferably equal to or more than 95% w/w, more preferably equal to or more than 99% w/w, yet more preferably equal to or more than 99.5% w/w, still more preferably equal to or more than 99.9% w/w and most preferably still more preferably equal to or more than 100.0% w/w of the bacterial particles have a particle size, suitably as determined by laser diffraction particle size analysis, diameter of equal to or less than 8.0 micrometer (μm), preferably equal to or less than 7.5 micrometer, more preferably equal to or less than 7.0 micrometer, yet more preferably equal to or less than 6.5 micrometer and most preferably equal to or less than 6.0 micrometer preferably equal or less than 5.5, 5.0, 4.5, 4.0, 3.5, 3.0, 2.5, or 2.0 micrometer, and the composition preferably comprises a cell viability of equal to or more than 2.00·10¹⁰ CFU/gram, more preferably a cell viability of equal to or more than 2.10·10¹⁰ CFU/gram, yet more preferably a cell viability of equal to or more than 1.50·10¹¹ CFU/gram and most preferably a cell viability of equal to or more than 2.00·10¹¹ CFU/gram.

In a sixth aspect, the invention provides a composition for animal and/or human consumption, preferably a medicament or a food product or a beverage product, comprising:
the Lactobacillus rhamnosus cells, respectively Lactobacillus rhamnosus bacterial particles, as referred to above; or
the composition comprising Lactobacillus rhamnosus cells, respectively Lactobacillus rhamnosus bacterial particles, as referred to above.

In a seventh aspect, the invention provides a use of the Lactobacillus rhamnosus cells, respectively Lactobacillus rhamnosus bacterial particles, as referred to above or the composition comprising Lactobacillus rhamnosus cells, respectively Lactobacillus rhamnosus bacterial particles, as referred to above in the production of a food or beverage product.

In an eighth aspect, the invention provides a use of the Lactobacillus rhamnosus cells, respectively Lactobacillus rhamnosus bacterial particles, as referred to above or the composition comprising Lactobacillus rhamnosus cells, respectively Lactobacillus rhamnosus bacterial particles, as referred to above in a probiotic composition and/or for medical purposes and/or in or as a medicament, preferably for use as a medicament for the treatment of or for prevention of a disease or condition in or related to the animal or human gastro-intestinal tract.

With the processes according to the invention and in the compositions and uses according to the invention the bacterial surface per gram product can be increased and/or the bacterial particle size can be decreased and/or a the count of CFU/gram can be increased and/or viable cell count can be increased. As illustrated in the examples the processes according to the invention lead to a synergetic effect resulting in a high count of CFU/gram and a small particle size. Together the high count of CFU/gram and the small particle size lead to a high overall bacterial particle surface area per gram product. In addition, without wishing to be bound by any kind of theory, it is believed that as a result of the processes applied, the pili on the cells may become less intertangled and more bioavailable. Without wishing to be bound by any kind of theory it is believed that such high overall cell surface area and associated high bioavailability of the pili on the cell surface may be beneficial when applied in the human or animal gastro-intestinal tract. Without wishing to be bound by any kind of theory it is believed that for example adherence to and/or interactions with human or animal intestinal epithelial cells may be increased, for example via the pili on the cell surface of the Lactobacillus rhamnosus cells. That is, without wishing to be bound by any kind of theory it is believed that the processes, compositions and uses according to the invention, resulting in and/or having the above mentioned high cell surface area, small particle size, high count of CFU/gram and/or high viable cell count may be beneficial for the probiotic activity of the Lactobacillus rhamnosus cells.

### BRIEF DESCRIPTION OF THE DRAWINGS

The invention is illustrated by the following FIGURES:

FIG. 1 shows the bacterial particle size distribution of Lactobacillus rhamnosus GG for the samples derived from fermentors B1 and C1 as illustrated in the examples. As illustrated, compared with the situation without shear (“light grey line”), the application of shear (“dark grey line”) allows for a substantial reduction in % volume with a particle size of more than 5 micrometer (μm) and the second peak of this line for fermentor B1 (according to the invention) is substantially lower (just above 2%), than the second peak of this line for fermentor C1 (comparative, just above 2.5%).

### DETAILED DESCRIPTION OF THE INVENTION

### Definitions

Unless defined otherwise or clearly indicated by context, all technical and scientific terms used herein have the same meaning as commonly understood by one of ordinary skill in the art.

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element. When referring to a noun (e.g. a compound, an additive, etc.) in the singular, the plural is meant to be included. Thus, when referring to a specific moiety, e.g. a “strain”, this means “at least one” of that strain, e.g. “at least one strain”, unless specified otherwise.

When referring to a compound of which several isomers exist (e.g. a D and an L enantiomer), the compound in principle includes all enantiomers, diastereomers and cis/trans isomers of that compound that may be used in the particular aspect of the invention; in particular when referring to such as compound, it includes the natural isomer(s).

Unless explicitly indicated otherwise, the various embodiments of the invention described herein can be cross-combined.

The term “milk” is intended to encompass milks from mammals and plant sources or mixtures thereof. Mammals sources of milk include, but are not limited to cow, sheep, goat, buffalo, camel, llama, horse or reindeer. Plant sources of milk include, but are not limited to, milk extracted from soy bean, pea, peanut, barley, rice, oat, quinoa, almond, cashew, coconut, hazelnut, hemp, sesame seed and sunflower seed. In addition, the term “milk” refers to not only whole milk, but also skim milk or any liquid component derived thereof or reconstituted milk.

Lactobacillus rhamnosus

The term lactic acid bacteria (LAB) is a general term for a class of non-spore forming, gram-positive bacteria whose main product of fermented sugar is lactic acid. Examples of probiotic bacteria include LAB bacteria of species of Lactobacillus, Bifidobacterium sp, and Saccharomyces. Lactobacillus rhamnosus is one of the most popular probiotic bacteria.

Preferably the Lactobacillus rhamnosus is one or more of Lactobacillus rhamnosus GG, Lactobacillus rhamnosus BD0016 and/or Lactobacillus rhamnosus KF 7.

More preferably the Lactobacillus rhamnosus in all the aspects of this invention is Lactobacillus rhamnosus GG. Lactobacillus rhamnosus GG is officially also referred to as Lacticaseibacillus rhamnosus GG. The terms are used herein interchangeably.

The term “Lactobacillus rhamnosus GG” is herein understood to refer to the Lactobacillus rhamnosus strain deposited at the American Type Culture Collection as ATCC 53103 by Sherwood Gorbach and Barry Goldin, or a mutant or variant thereof.

Most preferably the Lactobacillus rhamnosus in all the aspects of this invention is the Lactobacillus rhamnosus GG strain deposited as ATCC 53103.

The Lactobacillus rhamnosus may be present as Lactobacillus rhamnosus bacterial particles, where each bacterial particle may comprise one or more cells. Advantageously the processes according to the invention can allow one to reduce the bacterial particle size and increase the bacterial surface. Hence, preferably the Lactobacillus rhamnosus as present in the compositions according to the invention comprises or consists of Lactobacillus rhamnosus bacterial particles, which bacterial particles preferably comprise or consist of equal to or less than 100 cells per particle, more preferably equal to or less than 50 cells per particle, still more preferably equal to or less than 20 cells per particle, even more preferably equal to or less than 10 cells per particle and yet more preferably equal to or less than 5 cells per particle. Most preferably the Lactobacillus rhamnosus as present in the compositions according to the invention comprises or consists of Lactobacillus rhamnosus bacterial particles, which bacterial particles comprise or consist of equal to or less than 3 cells per particle.

### Step (a)

In step (a) of the processes according to the invention, the Lactobacillus rhamnosus GG is fermented in a lactose-free, preferably milk-free, medium in a fermentor, and a fermentation broth is removed from such fermentor.

The fermentor may comprise or consist of a fermentation reactor, also sometimes referred to as a fermentation vat or a fermentation tank. The fermentor may or may not comprise a bubbling system and be a bubble reactor; may or may not comprise a stirrer and be a stirred reactor; and/or may or may not comprise a loop and be a loop reactor. Combinations are also possible. When the fermentor comprises a stirrer, respectively is a stirred reactor, the stirrer is preferably operated at a stirring rate in the range from equal to or more than 1 round per minute (rpm), more preferably from equal to or more than 2 rounds per minute (rpm), yet more preferably from equal to or more than 5 rounds per minute (rpm), and even more preferably from equal to or more than 10 rounds per minute (rpm) to equal to or less than 500 rounds per minute (rpm), more preferably equal to or less than 300 rounds per minute (rpm), yet more preferably equal to or less than 200 rounds per minute (rpm), still more preferably equal to or less than 100 rounds per minute (rpm) and most preferably equal to or less than 50 rounds per minute (rpm).

Where the fermentor is a laboratory fermentor, the fermentor may comprise a volume in the range from equal to or more than 1 liter, more preferably equal to or more than 5 liter to equal to or less than 50 liters. More preferably the fermentor is an industrial fermentor. Hence, more preferably the fermentor is a fermentor having a volume of equal to or more than 50 liter, more preferably equal to or more than 100 liter, still more preferably equal to or more than 500 liter and most preferably equal to or more than 1000 liter and preferably equal to or less than 700000 liter, more preferably equal to or less than 500000 liter and even more preferably equal to or less than 250000 liter, still more preferably equal to or less than 100000 liter.

The Lactobacillus rhamnosus is suitably fermented in a medium in the fermentor. This medium may also be referred to herein as the fermentation medium. The medium can suitably be a solution, suspension or dispersion. Preferably the fermentation medium is an aqueous fermentation medium. Preferably the medium is an aqueous solution, suspension or dispersion. Where the fermentor is a laboratory fermentor, the fermentation medium may comprise a volume in the range from equal to or more than 1 liter, more preferably equal to or more than 5 liter to equal to or less than 50 liters. More preferably the fermentor is an industrial fermentor. Hence, more preferably the the fermentation medium has a volume of equal to or more than 50 liter, more preferably equal to or more than 100 liter, still more preferably equal to or more than 500 liter and most preferably equal to or more than 1000 liter and preferably equal to or less than 700000 liter, more preferably equal to or less than 500000 liter and even more preferably equal to or less than 250000 liter, still more preferably equal to or less than 100000 liter.

To allow use as a probiotic, also by lactose intolerant consumers, the medium has to be lactose-free. By lactose-free is herein understood that preferably the fermentation medium comprises equal to or less than 1000 ppmw (parts per million by weight) lactose, more preferably equal to or less than equal to or less than 100 ppmw lactose, even more preferably equal to or less than 10 ppmw lactose, yet more preferably equal to or less than 1 ppmw lactose and still more preferably equal to or less than 0.1 ppmw lactose. Most preferably the medium does not comprise any measurable lactose and is completely lactose free.

More preferably the medium is milk-deficient or milk-free. By milk-free is herein understood that preferably the fermentation medium comprises equal to or less than 1000 ppmv (parts per million by volume) milk, more preferably equal to or less than equal to or less than 100 ppmv milk, even more preferably equal to or less than 10 ppmv milk, yet more preferably equal to or less than 1 ppmv milk and still more preferably equal to or less than 0.1 ppmv milk. Most preferably the medium does not comprise any measurable milk and is completely milk free.

As set out above, the term milk includes milk from a mammal source and/or a plant-based source. Hence, the fermentation medium preferably does not comprise any milk derived from cow, sheep, goat, buffalo, camel, llama, horse or reindeer and/or extracted from soy bean, pea, peanut, barley, rice, oat, quinoa, almond, cashew, coconut, hazelnut, hemp, sesame seed or sunflower seed.

Further details on the fermentation medium are provided below. As set out below, more preferably the fermentation medium is a solution, suspension or dispersion wherein the acetate is present as a disassociated or a non-disassociated calcium acetate salt and/or a disassociated or a non-disassociated ammonium acetate salt, most preferably as a disassociated or a non-disassociated ammonium acetate salt. Preferably the fermentation medium is stirrable, more preferably a stirrable liquid or slurry, and preferably the medium is not a solid or a gel. In addition, further details on the fermentation conditions are provided below.

Conveniently step (a) may produce, respectively result in, a fermentation broth. As indicated above, such fermentation broth may suitably be removed from the fermentor before applying step (b).

### Glucose

Preferably the medium, also referred to herein as fermentation medium, is a glucose-containing medium. The glucose can be added as solid or as a solution. Preferably the glucose is added to the medium (also referred to herein as fermentation medium) in the form of an aqueous solution or aqueous suspension or aqueous dispersion comprising glucose.

The glucose may for example be added to the medium (also referred to herein as fermentation medium) before the start of the fermentation and/or during the fermentation. If glucose is added during the fermentation such glucose is preferably added in a continuous manner, for example by in-line addition, for example via a loop reactor. Preferably the glucose is present in the medium, for example at the start of the fermentation and/or during fermentation, in a concentration in the range from equal to or more than 20 gram/kilogram (gr/kg), preferably from equal to or more than 60 gram/kilogram (gr/kg), more preferably from equal to or more than 80 gr/kg, yet more preferably from equal to or more than 100 gr/kg, still more preferably from equal to or more than 110 gr/kg, to equal to or less than 600 gr/kg, more preferably to equal to or less than 400 gr/kg, even more preferably to equal to or less than 300 gr/kg, still more preferably to equal to or less than 200 gr/kg, yet more preferably to equal to or less than 170 gr/kg and most preferably equal to or less than 150 gr/kg, wherein gr/kg refers to the weight in grams of glucose per total weight in kilograms of medium. In the above range the preference for the specific concentrations in gr/kg can be interchanged with a corresponding preference for gr/liter.

That is, preferably the glucose is present in the medium, for example at the start of the fermentation and/or during fermentation, in a concentration in the range from equal to or more than 2% w/w, preferably from equal to or more than 6% w/w, more preferably from equal to or more than 8% w/w, yet more preferably from equal to or more than 10% w/w, still more preferably from equal to or more than 11% w/w, to equal to or less than 60% w/w, more preferably to equal to or less than 40% w/w, even more preferably to equal to or less than 30% w/w, still more preferably to equal to or less than 20% w/w, yet more preferably to equal to or less than 17% w/w and most preferably equal to or less than 15% w/w, based on the total weight of medium. Where the glucose is added as a solution, suspension or dispersion, the above ranges apply to the weight of the amount of glucose “as such” therein, not to the weight of the solution, suspension or dispersion as a whole.

### Calcium

Preferably the fermentation in step (a) is further carried out in the presence of calcium or a calcium salt.

The calcium may be present as calcium element or as a salt. If the calcium is present as a salt, the calcium may be present in a disassociated form or a non-disassociated (i.e. an associated) form. That is, the calcium may for example be present as a Ca²⁺ cation.

Preferably the calcium is present or supplied to the medium (also referred to herein as fermentation medium) as a disassociated or a non-disassociated calcium salt. Hence, preferably the fermentation medium comprises calcium or a calcium salt, suitably a disassociated or a non-disassociated calcium salt.

Preferably the calcium salt is a halogenide salt of calcium, an organic acid salt of calcium or calciumhydroxide. In one preferred embodiment the calcium salt is an halogenide salt of calcium, more preferably calcium chloride, calcium iodide or calcium bromide or a mixture thereof. Most preferably the halogenide salt of calcium is calcium chloride. In another preferred embodiment the calcium salt is an organic acid salt of calcium, preferably an organic acid salt comprising in the range from 1 to 6 carbon atoms, more preferably an organic salt comprising in the range from 1 to 4 carbon atoms. Preferably the organic acid salt of calcium is selected from the group consisting of calcium carbonate, calcium acetate, calcium propionate, calcium butanoate, calcium citrate, calcium gluconate or a mixture thereof. Most preferably the organic acid salt of calcium is calcium carbonate.

Most preferably the calcium is present or supplied to the medium as a disassociated or a non-disassociated calcium chloride salt and/or a disassociated or a non-disassociated calcium carbonate salt and/or a disassociated or a non-disassociated calciumhydroxide salt.

Preferably the calcium is added to the medium in the form of an aqueous solution or aqueous suspension or aqueous dispersion comprising calcium, optionally as a Ca²⁺ cation or otherwise as a disassociated or a non-disassociated salt. Hence, preferably the fermentation medium comprises an aqueous solution or aqueous suspension or aqueous dispersion comprising calcium or a disassociated or a non-disassociated calcium chloride salt and/or a disassociated or a non-disassociated calcium carbonate salt and/or a disassociated or a non-disassociated calciumhydroxide salt, most preferably a disassociated or a non-disassociated calcium chloride salt.

The calcium may for example be added to the medium (also referred to herein as fermentation medium) before the start of the fermentation and/or during the fermentation. If calcium is added during the fermentation such calcium is preferably added in a continuous manner, for example by in-line addition, for example via a loop reactor. Preferably the calcium is present in the medium, for example at the start of the fermentation and/or during fermentation, in a concentration in the range from equal to or more than 10 milligram/kilogram (mg/kg), preferably from equal to or more than 20 mg/kg, more preferably from equal to or more than 30 mg/kg, still more preferably from equal to or more than 40 mg/kg, to equal to or less than 10 gr/kg, more preferably to equal to or less than 5 gr/kg, even more preferably to equal to or less than 1 gr/kg, still more preferably to equal to or less than 500 mg/kg, yet more preferably to equal to or less than 300 mg/kg and most preferably equal to or less than 200 mg/kg, wherein gr/kg, respectively mg/kg, refers to the weight in grams, respectively milligrams, of calcium per total weight in kilograms of medium. That is, preferably the calcium is present in the medium, for example at the start of the fermentation and/or during fermentation, in a concentration in the range from equal to or more than 10 ppmw (parts per million by weight), preferably from equal to or more than 20 ppmw, more preferably from equal to or more than 30 ppmw, still more preferably from equal to or more than 40 ppmw, to equal to or less than 10% w/w, more preferably to equal to or less than 5% w/w, even more preferably to equal to or less than 1.0% w/w, still more preferably to equal to or less than 500 ppmw, yet more preferably to equal to or less than 300 ppmw and most preferably equal to or less than 200 ppmw, based on the total weight of medium. A very high amount calcium may be less preferred as depending on the circumstances such may lead to precipitation. The risk of precipitation is highest with calcium carbonate. Calcium carbonate unfortunately has a very low solubility in pure water (about 15 mg/L, corresponding to about 0.0015% w/w at 25° C.). Therefore most preferably any calcium salt is not present as calcium carbonate. Preferably any calcium salt is calcium chloride. Most preferably the calcium may be added and/or present within the medium, for example at the start of the fermentation and/or during fermentation, in a concentration in the range from equal to or more than 0.0001% w/w, more preferably from equal to or more than 0.001% w/w, to equal to or less than 0.1% w/w, more preferably equal to or less than 0.05% w/w, based on the total weight of the medium. That is, most preferably the fermentation medium comprises in the range from equal to or more than 0.0001% w/w, more preferably from equal to or more than 0.001% w/w, to equal to or less than 0.1% w/w, more preferably equal to or less than 0.05% w/w of calcium, based on the total weight of the medium.

Where the calcium is added as a salt or in a solution, suspension or dispersion, the above ranges apply to the weight of the amount of calcium “as such” therein, not to the weight of the salt, solution, suspension or dispersion as a whole.

### Acetate

In step (a) the fermentation is carried out in the presence of acetate or a acetate salt.

The acetate is preferably present as a salt. If the acetate is present as a salt, the acetate may be present in a disassociated form or a non-disassociated (i.e. an associated) form. That is, the acetate may for example be present as an acetate anion. Such acetate anion may for example be represented with the chemical formula CH₃CO⁻₂, C₂H₃O⁻₂, or CH₃COO⁻.

Preferably the acetate is present or supplied to the medium (also referred to herein as fermentation medium) as a disassociated or a non-disassociated acetate salt. Hence, preferably the fermentation medium comprises an acetate salt, suitably a disassociated or a non-disassociated acetate salt.

In a preferred embodiment the acetate salt is an alkali metal or alkali earth metal salt of acetate. Preferably such alkali or alkaline earth metal salt of acetate is selected from the group consisting of sodium acetate, potassium acetate, calcium acetate, or a mixture thereof. Most preferably the alkali or alkaline earth metal salt of acetate is calcium acetate. In another preferred embodiment the acetate is present as ammonium acetate. Such ammonium acetate may for example be represented with the chemical formula NH₄CH₃CO₂

More preferably the acetate is present or supplied to the medium as a disassociated or a non-disassociated calcium acetate salt and/or a disassociated or a non-disassociated ammonium acetate salt, most preferably as a disassociated or a non-disassociated ammonium acetate salt.

Preferably the acetate is added to the medium in the form of an aqueous solution or aqueous suspension or aqueous dispersion comprising acetate, optionally as an acetate anion or otherwise as a disassociated or a non-disassociated salt. Hence, preferably the fermentation medium comprises an aqueous solution or aqueous suspension or aqueous dispersion comprising an ammonium acetate salt, suitably a disassociated or a non-disassociated ammonium acetate salt.

The acetate may for example be added to the medium (also referred to herein as fermentation medium) before the start of the fermentation and/or during the fermentation. If acetate is added during the fermentation such acetate is preferably added in a continuous manner, for example by in-line addition, for example via a loop reactor. Preferably the acetate is present in the medium, for example at the start of the fermentation and/or during fermentation, in a concentration in the range from equal to or more than 0.4 gram/kilogram (gr/kg), preferably from equal to or more than 1.0 gram/kilogram (gr/kg), more preferably from equal to or more than 2.0 gr/kg, yet more preferably from equal to or more than 3.0 gr/kg, still more preferably from equal to or more than 4.0 gr/kg, to equal to or less than 60 gr/kg, preferably to equal to or less than 40 gr/kg, more preferably to equal to or less than 30 gr/kg, even more preferably to equal to or less than 20 gr/kg, still more preferably to equal to or less than 10 gr/kg, yet more preferably to equal to or less than 8.0 gr/kg and most preferably equal to or less than 6.0 gr/kg, wherein gr/kg refers to the weight in grams of acetate per total weight in kilograms of medium. In the above range the preference for the specific concentrations in gr/kg can be interchanged with a corresponding preference for gr/liter.

That is, preferably the acetate is present in the medium, for example at the start of the fermentation and/or during fermentation, in a concentration in the range from equal to or more than 0.04% w/w, preferably from equal to or more than 0.10% w/w, more preferably from equal to or more than 0.20% w/w, yet more preferably from equal to or more than 0.30% w/w, still more preferably from equal to or more than 0.40% w/w, to equal to or less than 6% w/w, preferably to equal to or less than 4% w/w, more preferably to equal to or less than 3% w/w, even more preferably to equal to or less than 2% w/w, still more preferably to equal to or less than 1% w/w, yet more preferably to equal to or less than 0.80% w/w and most preferably equal to or less than 0.60% w/w, based on the total weight of medium. Acetate salts that have a low solubility in water, such as for example vitamin A acetate or tocopherol acetate, may be less preferred as depending on the circumstances such may lead to precipitation. More preferred are acetate salts with a good solubility in water, such as ammonium acetate, potassium acetate and/or sodium acetate. Ammonium acetate is most preferred. Without wishing to be bound to any kind of theory it is believed the use of ammonium acetate may have less osmotic effects than for example sodium acetate or potassium actetate. Most preferably the fermentation medium comprises in the range from equal to or more than 0.10% w/w, more preferably from equal to or more than 0.20% w/w, to equal to or less than 2.0% w/w, more preferably equal to or less than 1.0% w/w of acetate, preferably in the form of ammonium acetate, based on the total weight of the medium.

Where the acetate is added as a salt, a solution, suspension or dispersion, the above ranges apply to the weight of the amount of acetate “as such” therein, not to the weight of the salt, solution, suspension or dispersion as a whole.

### Other Components in the Fermentation Medium

As set out above, the fermentation medium is preferably a solution, suspension or dispersion, more preferably an aqueous solution, suspension or dispersion, wherein the acetate is present as a disassociated or a non-disassociated calcium acetate salt and/or a disassociated or a non-disassociated ammonium acetate salt, most preferably as a disassociated or a non-disassociated ammonium acetate salt.

In addition to the components already mentioned hereinbefore, the medium (also referred to herein as the fermentation medium) may comprise one or more further components. That is, optionally the fermentation in step (a) can be carried out in the presence of one or more further components.

Preferably the medium comprises further components such as for example:
nutrients such as yeasts; and/or
pH adjusters such as ammoniumhydroxide, sodiumhydroxide or potassiumhydroxide; and/or
minerals such as manganese, magnesium and/or zinc and/or any associated or disassociated salt thereof.

More preferably the medium at least comprises one or more nutrients such as a yeast and/or a pH adjuster such as ammoniumhydroxide, sodiumhydroxide and/or potassiumhydroxide.

Most preferably the medium (also referred to herein as the fermentation medium) comprises sodiumhydroxide and/or potassiumhydroxide, preferably as pH adjuster. More preferably no ammonia (NH3) is added or otherwise supplied to the medium. That is, more preferably no ammonia (NH3) is used as pH adjuster. Most preferably sodium hydroxide is used as a pH adjuster. As described for example in European patent EP2398890B1 the use of ammonia as a titrant is understood to cause an unwanted pink/red color observed on the surface of dried lactic acid bacteria compositions during storage. Without wishing to be bound by any kind of theory it is believed that the use of NaOH and KOH base to control pH, as mentioned in EP2398890B1, may negatively impact cell counts. It has advantageously been found that such negative impact can be at least partly countered by applying an ammonium acetate salt in the fermentation.

Preferably the medium is an aqueous medium. In addition to the above, the medium therefore most preferably also comprises water.

### Fermentation Conditions, pH and Temperature

Preferably the fermentation is carried out at a temperature equal to or more than 10° C., more preferably equal to or more than 15° C., still more preferably equal to or more than 20° C., even more preferably equal to or more than 25° C., yet more preferably equal to or more than 28° C., still even more preferably equal to or more than 32° C. and most preferably equal to or more than 35° C. At the same time, the fermentation is preferably carried out at a temperature equal to or less than 46° C., more preferably equal to or less than 42° C., still more preferably equal to or less than 40° C., even more preferably equal to or less than 39° C., yet more preferably equal to or less than 38° C. and most preferably equal to or less than 37° C.

Preferably the fermentation is carried out at a pH point, or the fermentation is preferably directed towards an end pH point, that is equal to or more than pH 3.0, more preferably equal to or more than pH 3.5, still more preferably equal to or more than pH 4.2, even more preferably equal to or more than pH 4.6, yet more preferably equal to or more than pH 4.8 and most preferably equal to or more than pH 5.0. At the same time, the fermentation is preferably carried out at a pH point, or the fermentation is preferably directed towards an end pH point, that is equal to or less than pH 6.2, more preferably equal to or less than pH 6.0, still more preferably equal to or less than pH 5.8, even more preferably equal to or less than pH 5.6, yet more preferably equal to or less than pH 5.5 and most preferably equal to or less than pH 5.4.

The pH can conveniently be controlled by addition of a titrant, preferably a titrant selected from the group consisting of sodium hydroxide, potassium hydroxide, ammonium hydroxide and combinations thereof, as described herein. Suitably the use of sodium hydroxide and/or potassium hydroxide as titrant eliminates the need to use ammonia (NH3) as a titrant, and hence allows one to reduce pinking. Hence most preferably the titrant does not comprise ammonia (NH3). Most preferably the titrant comprises or consists of sodium hydroxide. Preferably the titrant is supplied to the fermentor as an aqueous solution of such titrant. That is, preferably an aqueous solution of potassium hydroxide and/or sodium hydroxide, most preferably an aqueous solution of sodium hydroxide is supplied to the fermentor.

By fermentation is herein preferably understood the process until the fermentation broth is removed from the fermentor. The fermentation can be a continuous, semi-continuous or batch-wise fermentation. If the fermentation is carried out batch-wise, the fermentation is preferably stopped by cooling, pH drop and/or depletion of the carbon source used as a feed whereafter conveniently the fermentation broth may be removed from the fermentor.

In a preferred embodiment, during the fermentation in step (a), the pH is maintained in the range from equal to or more than 5.4 to equal to or less than 5.6, whilst the temperature is maintained in the range from equal to or more than 37° C. to equal to or less than 39° C.

In a first preferred embodiment, the pH of the fermentation medium is maintained at 5.6, whilst the temperature is maintained at 39° C. In a second embodiment, the pH is maintained at 5.4, whilst the temperature is maintained at 39° C. In a third embodiment, the pH is maintained at 5.6, whilst the temperature is maintained at 39° C. In a fourth embodiment, the pH is maintained at 5.4, whilst the temperature is maintained at 37° C.

### Step (b)

In step (b) of the processes according to the invention, shear is applied to the fermentation broth. As indicated above, the shear is suitably applied to a fermentation broth that has been removed from the fermentor. The processes according to the invention may comprise additional steps between step (a) and step (b). However, preferably step (b) is carried out on the fermentation broth as produced and/or obtained by step (a).

The shear may advantageously be applied by mechanical treatment of the fermentation broth. Such mechanical treatment may for example comprise the subjection of the fermentation broth to a volumetric power input of 1-500 kW/m3, more preferably 1-200 kW/m3, even more preferably 1-100 kW/m3, preferably for a duration of 0.1-60 min, more preferably 1-30 min, and even more preferably 1-10 min. The mechanical treatment may preferably comprise or consist of a treatment with a mixer and/or a treatment with an homogenizer, and/or a treatment with a mill and/or a treatment with a centrifuge (including for example continuous centrifuges with or without differential gravity). More preferably step (b) comprises or consists of the application and/or addition of mechanical shearing stress to the fermentation broth, preferably by one of the above exemplified mechanical treatments.

In mechanics, shear forces are a common phenomenon. A shear force is understood to exist when there is a first force acting on (part of) a composition in a first direction, and a second force acting on (part of) the composition that is stationary or moving in a second non-aligned direction.

Shear stress (often denoted by the Greek symbol “tau” or “τ”) is the component of stress coplanar with a material cross section. It arises from the shear force and more specifically from the component of force vector parallel to the material cross section. Average shear stress refers to the force applied per unit area and can be calculated with formula (I) below:

$$τ = F / A \qquad ( I )$$

wherein:
   τ=the average shear stress;
   F=the force applied;
   A=the cross-sectional area of material with area parallel to the applied force vector.

Shear rate is the rate at which a progressive shearing deformation is applied. For a simple case, this can be the gradient of velocity in a flowing material. Shear rate is expressed in “reciprocal seconds, i.e. in “s⁻¹”.

In their article titled “The determination of viable counts in probiotic cultures microencapsulated by spray-coating”, published in Food Microbiology, volume 24, 2010, pages 1104-1111, Champagne et al. describe that high-shear homogenization (HSH) can be applied as part of an analytical method to determine viable counts (CFU) in freeze-dried and dried micro-encapsulated (ME) probiotic cultures. Microencapsulation was done by spray-coating of dried Lactobacillus rhamnosus R0011 or Bifidobacterium longum ATCC 15708 cultures with fat. They concluded that HSH reduced the variability of the CFU results of both free-cell and ME. However, in their subsequent article titled “Recommendations for the viability assessment of probiotics as concentrated cultures and in food matrices” published in Int. J. Food Microbioly, volume 149, 2011, pages 185-193, Champagne et. al, warn that too much homogenization may actually kill the cells. This is in line with other publications where high pressure, respectively high shear, homogenizers are actually advertised for disruption of cells.

In view of the above, the advantageous effects found by the inventors for the currently claimed processes are surprising.

In the processes according to the invention the shear is applied to the fermentation broth after such fermentation broth has been removed from the fermentor and preferably before such fermentation broth has optionally been frozen, freeze-dried or encapsulated.

Preferably a shear rate is applied in the range from equal to or more than 0.5 s⁻¹, preferably from equal to or more than 1.0 s⁻¹, more preferably from equal to or more than 10 s⁻¹, yet more preferably from equal to or more than 100 s⁻¹, still more preferably from equal to or more than 500 s⁻¹, to equal to or less than 500000 s⁻¹, preferably equal to or less than 100000 s⁻¹, more preferably equal to or less than 50000 s⁻¹, yet more preferably equal to or less than 10000 s⁻¹, still more preferably equal to or less than 5000 s⁻¹.

Preferably a shear stress is applied in the range from equal to or more than 1·10⁻⁹ Pascal, preferably from equal to or more than 1·10⁻⁸ Pascal, more preferably from equal to or more than 1·10⁻⁷ Pascal Pascal, yet more preferably from equal to or more than 1·10⁻⁶ Pascal, still more preferably from equal to or more than 1·10⁻⁵ Pascal, to equal to or less than 1.10-1 Pascal, more preferably equal to or less than 1·10⁻² Pascal, yet more preferably equal to or less than 1·10⁻³ Pascal, still more preferably equal to or less than 1·10⁻⁴ Pascal.

Shear can be applied to the fermentation broth in every manner known by the skilled person to be suitable therefore. Preferably the shear is applied to the fermentation broth by means of a homogenizer and/or centrifuge. More preferably the shear is applied by subjecting the fermentation broth to a centrifugation step. That is, preferably step (b) comprises or consists of centrifugating the fermentation broth. Centrifugation advantageously allows for simultaneous concentration of the fermentation broth. After centrifugation it can be advantageous to still apply further concentration steps, for example subsequent to centrifugation the fermentation broth can be filtered. However, advantageously, such filtration is not needed and step (b) can be carried out without filtration.

As indicated above, in a preferred embodiment step (b) may or may not comprise or consist of homogenization of the fermentation broth. Preferably step (b) comprises or consists of homogenization of the fermentation broth. If applied, homogenization can be applied to the fermentation broth before or after any optional concentration. Preferably any homogenization is applied before any optional (subsequent) concentration. When step (b) comprises or consists of homogenization of the, preferably unconcentrated, fermentation broth, homogenization is preferably carried out at a rate in the range from equal to or more than 1000 rounds per minute (rpm), more preferably from equal to or more than 3000 rounds per minute (rpm), yet more preferably from equal to or more than 5000 rounds per minute (rpm), still more preferably from equal to or more than 8000 rounds per minute (rpm), even still more preferably from equal to or more than 10000 rounds per minute (rpm), yet even more preferably from equal to or more than 11000 rounds per minute (rpm), most preferably from equal to or more than 12000 rounds per minute (rpm), to equal to or less than 50000 rounds per minute (rpm), more preferably equal to or less than 30000 rounds per minute (rpm), yet more preferably equal to or less than 23000 rounds per minute (rpm), still more preferably equal to or less than 18000 rounds per minute (rpm) and most preferably equal to or less than 16000 rounds per minute (rpm). When step (b) comprises or consists of homogenization of the, preferably unconcentrated, fermentation broth, homogenization is preferably carried out for a period (also referred to as duration) in the range from equal to or more than 0.5 minute, more preferably from equal to or more than 1 minute, yet more preferably from equal to or more than 2 minutes, and still more preferably from equal to or more than 3 minutes, yet still more preferably from equal to or more than 4 minutes to equal to or less than 30 minutes, more preferably equal to or less than 20 minutes, yet more preferably equal to or less than 10 minutes, still more preferably equal to or less than 7 minutes and most preferably equal to or less than 5 minutes.

In a most preferred embodiment step (b) comprises or consists of homogenization of an unconcentrated fermentation broth, wherein such homogenization is carried out at a rate in the range from more than 10000 rounds per minute (rpm), more preferably from equal to or more than 11000 rounds, most preferably from equal to or more than 12000 rounds per minute (rpm), to equal to or less than 50000 rounds per minute (rpm), more preferably equal to or less than 30000 rounds per minute (rpm), for a period (also referred to as duration) in the range from equal to or more than 1 minute, yet more preferably from equal to or more than 2 minutes, and still more preferably from equal to or more than 3 minutes, yet still more preferably from equal to or more than 4 minutes to equal to or less than 30 minutes, more preferably equal to or less than 20 minutes, yet more preferably equal to or less than 10 minutes, still more preferably equal to or less than 7 minutes and most preferably equal to or less than 5 minutes.

In another preferred embodiment step (b) may or may not comprise or consist of centrifugation of the fermentation broth. When step (b) comprises or consists of centrifugation of the fermentation broth, centrifugation is preferably carried out at a centrifugation rate in the range from equal to or more than 500 rounds per minute (rpm), more preferably from equal to or more than 800 rounds per minute (rpm), yet more preferably from equal to or more than 1000 rounds per minute (rpm), still more preferably from equal to or more than 1500 rounds per minute (rpm), and most preferably from equal to or more than 2000 rounds per minute (rpm), to equal to or less than 30000 rounds per minute (rpm), more preferably equal to or less than 20000 rounds per minute (rpm), yet more preferably equal to or less than 16000 rounds per minute (rpm), still more preferably equal to or less than 10000 rounds per minute (rpm) and most preferably equal to or less than 8000 rounds per minute (rpm). When step (b) comprises or consists of centrifugation of the fermentation broth, centrifugation is preferably carried out for a period (also referred to as duration) in the range from equal to or more than 0.5 minute, more preferably from equal to or more than 1 minute, yet more preferably from equal to or more than 2 minutes, and still more preferably from equal to or more than 3 minutes, to equal to or less than 30 minutes, more preferably equal to or less than 20 minutes, yet more preferably equal to or less than 10 minutes, still more preferably equal to or less than 7 minutes and most preferably equal to or less than 5 minutes.

Preferably step (b) also comprises concentration of the fermentation broth. Concentration can be carried out by any means known to the skilled person to be suitable therefore. Preferably concentration is carried out by means of filtration and/or centrifugation. That is, preferably step (b) comprises filtration and/or centrifugation. When concentrated, the fermentation broth is preferably concentrated by a concentration factor of, or until a concentration factor is reached of, equal to or more than 2, more preferably equal to or more than 5, yet more preferably equal to or more than 7, even more preferably equal to or more than 8, still more preferably equal to or more than 10, and most preferably equal to or more than 12. The fermentation broth may suitably be concentrated by a factor of equal to or less than 1000, more suitably equal to or less than 200, even more suitably equal to or less than 100. Most preferably step (b) comprises concentration of the fermentation broth, most preferably by centrifugation, and the fermentation broth is concentrated until a concentration factor is reached of equal to or more than 10, more preferably equal to or more than 12.

In a further preferred embodiment step (b) may comprise or consist of homogenization of the fermentation broth, optionally filtration of the fermentation broth, and subsequent centrifugation of the, suitably homogenized, fermentation broth. Preferences for the homogenization and centrifugation are as indicated above. When step (b) comprises or consists of homogenization and centrifugation of the fermentation broth, centrifugation preferably allows for a further concentration of the fermentation broth as indicated above. Most preferably such further concentration is carried out until a concentration factor is reached of equal to or more than 8, more preferably equal to or more than 10 and even more preferably equal to or more than 12.

Step (b) may suitably result in a sheared and/or concentrated fermentation broth, more preferably in an homogenized and/or filtrated and/or centrifugated fermentation broth.

Preferably the Lactobacillus rhamnosus cells produced, obtained or obtainable by step (b) are Lactobacillus rhamnosus cells, wherein equal to or more than 90% w/w, preferably equal to or more than 95% w/w, more preferably equal to or more than 99% w/w, yet more preferably equal to or more than 99.5% w/w, still more preferably equal to or more than 99.9% w/w and most preferably still more preferably equal to or more than 100.0% w/w of the bacterial particles have a particle size, as determined by laser diffraction particle size analysis, diameter of equal to or less than 8.0 micrometer (μm), preferably equal to or less than 7.5 micrometer, more preferably equal to or less than 7.0 micrometer, yet more preferably equal to or less than 6.5 micrometer and most preferably equal to or less than 6.0 micrometer preferably equal or less than 5.5, 5.0, 4.5, 4.0, 3.5, 3.0, 2.5, or 2.0 micrometer, and the cells preferably have a cell viability of equal to or more than 2.00·10¹⁰ CFU/gram, more preferably a cell viability of equal to or more than 2.10·10¹⁰ CFU/gram, yet more preferably, for example by means of applying the concentration step, a cell viability of equal to or more than 1.50·10¹¹ CFU/gram and most preferably a cell viability of equal to or more than 2.00·10¹¹ CFU/gram.

In view of the above, step (b) thus preferably produces a composition comprising Lactobacillus rhamnosus cells, wherein equal to or more than 90% w/w, preferably equal to or more than 95% w/w, more preferably equal to or more than 99% w/w, yet more preferably equal to or more than 99.5% w/w, still more preferably equal to or more than 99.9% w/w and most preferably still more preferably equal to or more than 100.0% w/w of the bacterial particles have a particle size, as determined by laser diffraction particle size analysis, diameter of equal to or less than 8.0 micrometer (μm), preferably equal to or less than 7.5 micrometer, more preferably equal to or less than 7.0 micrometer, yet more preferably equal to or less than 6.5 micrometer and most preferably equal to or less than 6.0 micrometer preferably equal or less than 5.5, 5.0, 4.5, 4.0, 3.5, 3.0, 2.5, or 2.0 micrometer, and the composition preferably comprises a cell viability of equal to or more than 2.00·10¹⁰ CFU/gram, more preferably a cell viability of equal to or more than 2.10·10¹⁰ CFU/gram, yet more preferably, for example by means of applying the concentration step, a cell viability of equal to or more than 1.50·10¹¹ CFU/gram and most preferably a cell viability of equal to or more than 2.00·10¹¹ CFU/gram.

### Other Process Steps

As indicated above, step (b) may suitably result in a sheared and/or concentrated fermentation broth, more preferably in an homogenized and/or filtrated and/or centrifugated fermentation broth.

In addition to steps (a) and (b) the process preferably comprises one or more additional process steps. More preferably the process comprises a freezing and/or drying step.

Hence, the invention also provides a process for the production of a Lactobacillus rhamnosus, respectively Lactobacillus rhamnosus cells, respectively a composition comprising a Lactobacillus rhamnosus, respectively Lactobacillus rhamnosus cells, wherein the process comprises the steps of:
(a) fermenting the Lactobacillus rhamnosus in a lactose-free, preferably milk-free, medium in a fermentor, and removing a fermentation broth from the fermentor, wherein the fermentation is carried out in the presence of acetate or a acetate salt;
(b) applying shear to the fermentation broth and concentrating the fermentation broth; and
(c) freezing and/or drying the concentrated fermentation broth.

Methods for freezing and/or drying of an optionally concentrated fermentation broth are known in the art. For example, suitable drying methods include vacuum drying, infrared convection drying, microwaving, freeze-drying and/or spray-drying. Combinations of these are also possible.

Preferably step (c) comprises spray-drying or freeze-drying of the concentrated fermentation broth. If step (c) comprises freeze-drying, step (c) preferably comprises freezing and subsequent freeze-drying of a sheared and concentrated fermentation broth, resulting from, respectively produced or obtained by, step (b). Preferably step (b) results in, respectively produces an, an homogenized and/or filtrated and/or centrifugated fermentation broth. Hence, preferably step (c) comprises spray-drying; freezing; or freezing and freeze-drying of an homogenized and/or filtrated and/or centrifugated fermentation broth resulting from, respectively produced or obtained by, step (b).

During the above processes, preferably after step (a) but before step (c) one or more additives, preferably cryoprotectants and/or stabilizers, may be added. More preferably such cryoprotectants and/or stabilizers are added to the sheared and concentrated fermentation broth, preferably to an homogenized and/or filtrated and/or centrifugated fermentation broth. Such sheared and concentrated fermentation broth, preferably homogenized and/or filtrated and/or centrifugated fermentation broth, is conveniently resulting from, respectively produced or obtained by, step (b). Hence preferably any cryoprotectants and/or stabilizers are added subsequent to step (b) and before step (c). However, if so desired, any cryoprotectants and/or stabilizers may also be added after step (a) and before step (b).

Preferred cryoprotectants or stabilizers include, but are not limited to, glucose, lactose, raffinose, sucrose, trehalose, adonitol, starch maltodextrin, glycerol, mannitol sorbitol, plolypropylene glycol, polyethylene glycol, ribitol alginate, bovine serum albumin, carnitine, citrate, cystein, dextran, dimethyl sufoxide, sodium glutamate, glycin, betaine, glycogen, hypotaurin, skimmed milk peptone, polyvinyl pirrolidine, taurine, nucleosides, nucleotides and any combinations thereof.

### Compositions

The invention further provides the following compositions:

Lactobacillus rhamnosus bacterial particles or a composition comprising Lactobacillus rhamnosus bacterial particles, preferably obtained or obtainable by the processes described above.

Lactobacillus rhamnosus bacterial particles, wherein preferably equal to or more than 90% w/w, preferably equal to or more than 95% w/w, more preferably equal to or more than 99% w/w, yet more preferably equal to or more than 99.5% w/w, still more preferably equal to or more than 99.9% w/w and most preferably still more preferably equal to or more than 100.0% w/w of the bacterial particles have a particle size, as suitably determined by laser diffraction particle size analysis, of equal to or less than 8.0 micrometer, preferably equal to or less than 7.5 micrometer, more preferably equal to or less than 7.0 micrometer, yet more preferably equal to or less than 6.5 micrometer and most preferably equal to or less than 6.0 micrometer, preferably equal or less than 5.5, 5.0, 4.5, 4.0, 3.5, 3.0, 2.5, or 2.0 micrometer, and the bacterial particles preferably have a cell viability, respectively viable cell count, of equal to or more than 2.00·10¹⁰ CFU/gram, more preferably of equal to or more than 2.10·10¹⁰ CFU/gram, yet more preferably of equal to or more than 1.50·10¹¹ CFU/gram and most preferably of equal to or more than 2.00·10¹¹ CFU/gram.

A composition comprising Lactobacillus rhamnosus bacterial particles, wherein preferably equal to or more than 90% w/w, preferably equal to or more than 95% w/w, more preferably equal to or more than 99% w/w, yet more preferably equal to or more than 99.5% w/w, still more preferably equal to or more than 99.9% w/w and most preferably still more preferably equal to or more than 100.0% w/w of the bacterial particles have a particle size, as suitably determined by laser diffraction particle size analysis, of equal to or less than 8.0 micrometer, preferably equal to or less than 7.5 micrometer, more preferably equal to or less than 7.0 micrometer, yet more preferably equal to or less than 6.5 micrometer and most preferably equal to or less than 6.0 micrometer, preferably equal or less than 5.5, 5.0, 4.5, 4.0, 3.5, 3.0, 2.5, or 2.0 micrometer, and the composition preferably comprises cell viability, respectively viable cell count, of equal to or more than 2.00·10¹⁰ CFU/gram, more preferably of equal to or more than 2.10·10¹⁰ CFU/gram, yet more preferably of equal to or more than 1.50·10¹¹ CFU/gram and most preferably of equal to or more than 2.00·10¹¹ CFU/gram.

In an especially preferred embodiment the Lactobacillus rhamnosus bacterial particles have a particle size distribution, as suitably determined by laser diffraction particle size analysis, wherein the particle size distribution is bimodal. That is, preferably such particle size distribution has two peaks, a first peak (“peak 1”) in particles with a particle size equal to or smaller than 2.0 micrometer and a second peak (“peak 2”) in particles having a particle size equal to or larger than 2.0 micrometer. That is, the Lactobacillus rhamnosus bacterial particles preferably have a biomodal particle size distribution, as suitably determined by laser diffraction parties size analysis, wherein a first peak (“peak 1”) is situationed before 2.0 micrometer and a second peak (“peak 2”) is situated after 2.0 micrometer. More preferably the ratio of the peak area of peak 1 to the peak area of peak 2 is a ratio in the range from 3:1 to 5:1, more preferably about 4:1. That is, the Lactobacillus rhamnosus bacterial particles preferably have a biomodal particle size distribution, as suitably determined by laser diffraction parties size analysis, wherein equal to or more than 75%, preferably equal to or more than 76%, more preferably equal to or more than 77%, even more preferably equal to or more than 78% w/w, yet more preferably equal to or more than 79%, still more preferably equal to or more than 80%, yet still more preferably equal to or more than 81%, even still more preferably equal to or more than 82% and most preferably preferably equal to or more than 83% of the particles, have a particle size of equal to or less than 2.0 micrometer. Preferably the remaining Lactobacillus rhamnosus bacterial particles in the biomodal particle size distribution, i.e. the particles that have a particle size of more than 2.0 micrometer in the biomodal particle size distribution, have a particle size distribution wherein equal to or more than 90%, preferably equal to or more than 95%, more preferably equal to or more than 99%, yet more preferably equal to or more than 99.5%, still more preferably equal to or more than 99.9% and most preferably still more preferably equal to or more than 100.0% of these bacterial particles (i.e. these particles having a particles size of more than 2.0 micrometer) have a particle size, as suitably determined by laser diffraction particle size analysis, of equal to or less than 8.0 micrometer, preferably equal to or less than 7.5 micrometer, more preferably equal to or less than 7.0 micrometer, yet more preferably equal to or less than 6.5 micrometer and most preferably equal to or less than 6.0 micrometer.

Preferably the above compositions further comprise one or more additives, preferably cryoprotectants and/or stabilizers. Preferred cryoprotectants or stabilizers include, but are not limited to, glucose, lactose, raffinose, sucrose, trehalose, adonitol, starch maltodextrin, glycerol, mannitol sorbitol, plolypropylene glycol, polyethylene glycol, ribitol alginate, bovine serum albumin, carnitine, citrate, cystein, dextran, dimethyl sufoxide, sodium glutamate, glycin, betaine, glycogen, hypotaurin, skimmed milk peptone, polyvinyl pirrolidine, taurine, nucleosides, nucleotides and any combinations thereof.

Preferably the compositions are lactose-free composition, more preferably the compositions are milk-free compositions. Preferably the compositions may further comprise acetate or a acetate salt.

### Uses

The invention further provides the following uses and applications:

A composition for animal and/or human consumption, preferably a medicament or a food product or a beverage product, comprising:
the Lactobacillus rhamnosus bacterial particles as described above; or
the composition comprising Lactobacillus rhamnosus bacterial particles as described above.

Use of the Lactobacillus rhamnosus bacterial particles as described above or the composition comprising Lactobacillus rhamnosus bacterial particles as described above in the production of a food or beverage product.

Use of the Lactobacillus rhamnosus bacterial particles as described above or the composition comprising Lactobacillus rhamnosus bacterial particles as described above in a probiotic composition and/or for medical purposes and/or in or as a medicament, preferably for use as a medicament for the treatment of or for prevention of a disease or condition in or related to the animal or human gastro-intestinal tract.

Within the context of the present invention, the term “food product” is intended to encompass any consumable matter. Hence, it may be a product intended for the consumption by humans, but the term also encompasses products to be consumed by animals

In a preferred embodiment, the food or beverage product is a dairy product, preferably a yoghurt, a cheese, a butter, a buttermilk, quark, a sour cream, a kefir, a twarog, a fermented whey-based beverage, a koumiss, a milk beverage, a yoghurt drink, a fermented milk, a matured cream, a fromage frais, a milk, a dairy product retentate, a processed cheese, a cottage cheese, a cream dessert, or infant milk.

In another preferred embodiment the composition obtained or obtainable by the process as described herein or the compositions as described herein may be added to other components to create composition for use as probiotics and/or prebiotics. They may be used a direct fed microbials in food or feed for humans or animals. The probiotics and/or prebiotics may be used as a food or feed additive for humans or animals.

In a further aspect, the invention provides for a probiotic comprising or consisting of Lactobacillus rhamnosus bacterial particles, respectively a composition comprising or consisting of Lactobacillus rhamnosus bacterial particles as described herein, and preferably acetate or a acetate salt.

In the context of the present invention, the term “probiotic” is intended to refer to any micro-organism that is wished to be consumed owing to any beneficial effect it may have on its consumer.

In a preferred embodiment, the composition obtained or obtainable by the process as described herein or the composition as described herein is a pharmaceutical composition, in such embodiments the composition optionally comprises a pharmaceutically acceptable excipient.

In yet a further aspect, the composition obtained or obtainable by the process as described herein or the composition or probiotic as described herein for use as a medicament, preferably for the treatment of gastro-intestinal disorders and/or to improve gut health, for example to improve symptoms of irritable bowel syndrome and/or to improve digestive health and/or strengthen the immune system.

In yet a further aspect, the invention provides for a use of the composition obtained or obtainable by the process as described herein or the composition as describe herein in a food or beverages product.

Hereinbelow the invention is illustrated by a non-limiting example.

### EXAMPLES

As illustrated in the example below, applying acetate in the medium during the fermentation and applying shear to the fermentation broth after the fermentation allows for a beneficial synergetic effect to occur, resulting in a small particle size and high count of CFU/gram and thus resulting in a high bioavailability of pili.

### Materials and Methods

### Method for Determining the Count of CFU (Colony Forming Units) Per Gram.

For enumeration of the count of CFU/gram Lactobacillus rhamnosus GG a pour plate technique was used.

In a first step 1 gram of fermentation broth was weighted and mixed it with 9 grams of sterile PSW (Peptone Salt Water (PSW) from Bio Trading). After the creation of the initial suspension, a decimal dilution range was made by pipetting 1 ml+/−0.01 ml of the initial suspension into a tube with 9 ml sterile PSW solution using a sterile disposable serological 2 ml pipette. For each dilution step a new pipette was used. It was ensured that there were no air bubbles present and that there were no drops falling out or sticking to the side of the pipette. Subsequently the total was mixed by vortexing until a homogenous suspension was obtained. The above steps were repeated until the desired dilutions were reached.

For the pour plate technique, 1 ml (+/−0.01 ml) of the desired dilution was transferred to a 90 mm sterile petri dish with a ridge. Hereafter, 10⁻²⁰ ml of molten and tempered agar (MRS with glucose) (46° C.+/−1° C.) was poured into the petri-dish. The petri-dishes were swirled to spread the inoculated material homogeneously throughout the agar. The agar was melted (this can for example be done by using a microwave, a 99° C. water bath or autoclave pan) and was tempered to 46+/−1° C. for at least 30 minutes prior to use. The agar was allowed to solidify and the petri dishes were incubated upside down at the appropriate conditions specific for 2-3 days at 37° C. The plates were protected during incubation against dehydration. Anaerobic conditions were obtained by incubating the petri dishes in an anaerobic jar with AnaeroGen packs (Oxoid) in accordance with the manufacturing's instructions. An CO₂ indicator strip was added for verification.

After incubation, the plates that contained between 25 and 250 colony forming units (CFU) were counted. The viable cell count data was expressed in CFU per ml or CFU per gram accordingly. Below only the CFU numbers on the plates were used.

### Method for Determining the Bacterial Particle Size Distribution

The bacterial particle size distribution of fermentation samples was analysed by using a Laser Diffraction Particle Size Analyzer (LS 13 320 by Beckman Coulter) in line with the provided manual. If so desired, the Fraunhofer optical model can be used for determining the bacterial particle size (μm). For all samples, the bacterial particle size was recorded over a range from 0.4 to 20 μm and the outcome was expressed as volume (%) whereby the sum of all size sizes added up to 100%. In each case 0.65 ml of fermentation broth was added to the sample port and the sample was subsequently analysed. The vessel contained 0.85% NaCl. A graph was generated wherein for all sample fractions the cell size was expressed on the x-axis ranging from 0.4 to 20 μm, the volume (%) was expressed on the y-axis.

### Example 1

A Lactobacillus rhamnosus GG strain (derived from deposit ATCC 53103 as mentioned above) was added to several (separate) fermentors. In each fermentor the Lactobacillus rhamnosus GG strain was fermented in an aqueous fermentation medium comprising the components as listed in Table 1 under the conditions as listed in Table 1. During the fermentation glucose as listed in Table 1 was used as (carbohydrate) feed and the Lactobacillus rhamnosus GG was allowed to ferment this glucose. When the glucose was depleted, samples were taken. At the moment of sample taking samples were split up in two fractions:
(i) The first fraction of each sample was frozen in liquid nitrogen;
(ii) The second fraction of each sample was subjected to the application of shear and subsequently frozen in liquid nitrogen. The shear was applied by mixing the fraction of the sample during 4 minutes in an Ultra-Turrax mixer at 13000 rpm.

For each fraction of each sample, the count of CFU/gram was determined as described under materials and methods. The results are provided in Table 2. As can be seen in Table 2, the application of acetate individually results in an improvement (Δ CFU/gram) of 0.33·10¹⁰ CFU/gr and the application of shear individually results in an improvement (Δ CFU/gram) of 0.48·10¹⁰. When the acetate and shear are applied together an increased synergistic effect is obtained where the improvement (Δ CFU/gram) was 0.97·10¹⁰ CFU/gr.

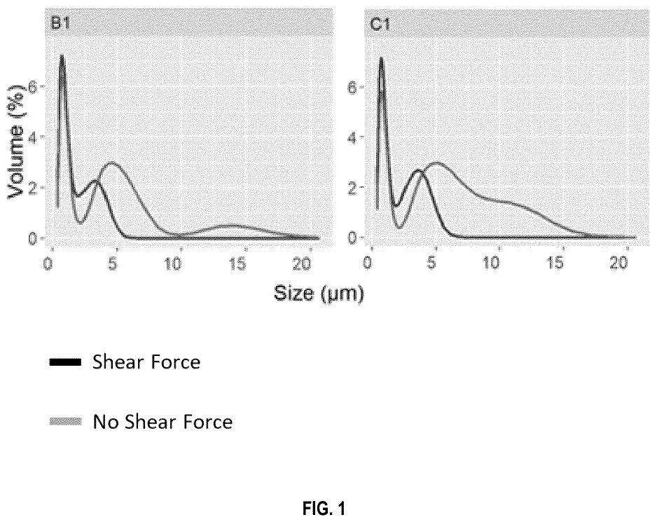

In addition, for each fraction of each sample, the bacterial particle size distribution was determined as described under materials and methods. The results are provided in FIG. 1. In FIG. 1 the bacterial particle size distribution is provided for the fraction without shear application (light grey lines) and the fraction after shear application (dark grey).

As can be seen in FIG. 1, in the reflection on fermentor 1, without application of acetate and shear force a large variation in bacterial particle size distribution exists (light grey lines), but after the application of acetate and Ultra Turrax treatment the bacterial particle size distribution has decreased. The overall bacterial particle size is much smaller after the application of acetate and shear which allows for a higher amount of bacterial particles per gram.

TABLE 1
Fermentation medium components and fermentation medium conditions.

| | Glucose | NH4-acetate | Yeast | Temp | | Calcium | Shear |
|---|---|---|---|---|---|---|---|
| Example | (gr/kg) | (gr/kg) | (gr/kg) | (° C.) | pH | (mg/kg)* | (rpm; min) |
| A (comp) (Fermentor B1) | 110 | 4.4 | 63.4 | 39 | 5.6 | Yes | n.a. |
| 1 (Fermentor B1) | 110 | 4.4 | 63.4 | 39 | 5.6 | Yes | 13000 |
| | | | | | | | 4 min. |
| B (comp) (Fermentor C1) | 110 | 0 | 63.4 | 39 | 5.6 | yes | n.a. |
| C (comp) (Fermentor C1) | 110 | 0 | 63.4 | 39 | 5.6 | yes | 13000 |
| | | | | | | | 4 min. |

*where calcium was present in the fermentation medium, such calcium was added at the start of the fermentation by means of an aqueoussolution with a concentration of about 55 to 56 mg calcium /kg. For each fermentor the same solution was added.

TABLE 2
Results for the count of CFU/gram,

| | | Δ CFU/gram compared to |
|---|---|---|
| Example | CFU/gram | comparative example “B” |
| A (comp) (Fermentor B1) | 1.54 · 10¹⁰ | 0.33 · 10¹⁰ |
| 1 (Fermentor B1) | 2.18 · 10¹⁰ | 0.97 · 10¹⁰ |
| B (comp) (Fermentor C1) | 1.21 · 10¹⁰ | 0.00 · 10¹⁰ |
| C (comp) (Fermentor C1) | 1.69 · 10¹⁰ | 0.48 · 10¹⁰ |

### Prophetic Example 2

The following is a prophetic example. A Lactobacillus rhamnosus GG strain (derived from deposit ATCC 53103 as mentioned above) is added to several (separate) fermentors. In each fermentor the Lactobacillus rhamnosus GG strain is fermented in an aqueous fermentation medium comprising glucose as a carbon hydrate feed and the Lactobacillus rhamnosus GG is allowed to ferment this glucose. The effect of the type of acetate in combination with titrants sodium hydroxide (NaOH) or potassium hydroxide (KOH) is investigated in pH-controlled fermentation of LbrGG at pH 5.6 and 39° C. The effects of both ammonium acetate (NH4-acetate) as well as sodium acetate (Na-acetate) are evaluated. The pH is controlled by the titrant and fermentation is stopped upon carbon depletion. Subsequently the pH in the fermenter is increased to 6.2 and the temperature in the fermenter is reduced to 7° C. When this temperature is reached, samples are harvested and subjected to the application of shear and subsequently frozen in liquid nitrogen. The shear is applied by mixing the sample during 4 minutes in an Ultra-Turrax mixer at 13000 rpm.

The cells counts for live, damaged and dead cells in the samples are determined by flow cytometry measurement (FCM). It is found that without an acetate source live cell counts were always lower when compared to the addition of an acetate source. As compared to the samples without an acetate source, for NaOH as titrant the live cells increase is about 35% when NH4-acetate is used and about 15% when Na-acetate is used as acetate source. A similar effect is found for KOH as titrant and also here NH4-acetate results in a higher number of live cells than Na-acetate.

1-19. (canceled)

20. A process for the production of bacterial particles of Lactobacillus rhamnosus or a composition comprising bacterial particles of Lactobacillus rhamnosus, wherein the process comprises the steps of:
(a) fermenting the Lactobacillus rhamnosus in a lactose-free, optionally milk-free, medium in a fermentor, and removing a fermentation broth from the fermentor, wherein the fermentation is carried out in the presence of ammonium;
(b) applying shear to the fermentation broth and optionally concentrating the fermentation broth, wherein when concentrating the fermentation broth, optionally comprising the addition of one or more additives, optionally cryoprotectants and/or stabilizers, to said concentrated fermentation broth.

21. The process according to claim 1, wherein the fermentation is carried out in the presence of ammonium acetate and wherein the fermentation is carried out at a pH point, or the fermentation is directed towards an end pH point, that is equal to or more than pH 3.0, wherein the pH is controlled by addition of a titrant, wherein the titrant is selected from the group consisting of sodium hydroxide and potassium hydroxide.

22. The process according to claim 1, wherein the fermentation medium is a solution, suspension or dispersion, optionally an aqueous solution, suspension or dispersion, wherein optionally acetate is present as a disassociated or a non-disassociated ammonium acetate salt.

23. The process according to claim 1, wherein the fermentation medium has a volume of equal to or more than 50 liter.

24. The process according to claim 1, wherein step (b) comprises homogenization of an unconcentrated fermentation broth, wherein such homogenization is carried out at a rate in the range from more than 10000 rounds per minute (rpm) to equal to or less than 50000 rounds per minute (rpm), for a period in the range from equal to or more than 1 minute to less than 5 minutes.

25. The process according to claim 1, wherein a shear rate is applied in the range from equal to or more than 0.5 s⁻¹, optionally from equal to or more than 1.0 s⁻¹, optionally from equal to or more than 10 s⁻¹, optionally from equal to or more than 100 s⁻¹, optionally from equal to or more than 500 s⁻¹, to equal to or less than 500000 s⁻¹, optionally equal to or less than 100000 s⁻¹, optionally equal to or less than 50000 s⁻¹, optionally equal to or less than 10000 s⁻¹, optionally equal to or less than 5000 s⁻¹.

26. The process according to claim 1, wherein a shear stress is applied in the range from equal to or more than 1·10⁻⁹ Pascal, optionally from equal to or more than 1·10⁻⁸ Pascal, optionally from equal to or more than 1·10⁻⁷ Pascal, optionally from equal to or more than 1·10⁻⁶ Pascal, optionally from equal to or more than 1·10⁻⁵ Pascal, to equal to or less than 1·10⁻¹ Pascal, optionally equal to or less than 1·10⁻² Pascal, optionally equal to or less than 1·10⁻³ Pascal, optionally equal to or less than 1·10⁻⁴ Pascal.

27. The process according to claim 1, wherein the shear is applied by subjecting the fermentation broth to a homogenization and/or a centrifugation step.

28. The process according to claim 1, wherein the process comprises the steps of:
(a) fermenting the Lactobacillus rhamnosus in a lactose-free, optionally milk-free, medium in a fermentor, and removing a fermentation broth from the fermentor, wherein the fermentation is carried out in the presence of ammonium acetate;
(b) applying shear to the fermentation broth and concentrating the fermentation broth; and
(c) freezing and/or drying the concentrated fermentation broth.

29. The process according to claim 1, wherein the process comprises spray-drying or freeze-drying of a concentrated fermentation broth.

30. Lactobacillus rhamnosus bacterial particles or a composition comprising Lactobacillus rhamnosus bacterial particles, obtained or obtainable by the process according to claim 1.

31. Lactobacillus rhamnosus bacterial particles or compositions comprising Lactobacillus rhamnosus bacterial particles obtained by the method according to claim 1, wherein equal to or more than 90% w/w, optionally equal to or more than 95% w/w, optionally equal to or more than 99% w/w, optionally equal to or more than 99.5% w/w, optionally equal to or more than 99.9% w/w and optionally equal to or more than 100.0% w/w of the bacterial particles have a particle size, as determined by laser diffraction particle size analysis, of equal to or less than 8.0 micrometer, optionally equal to or less than 7.5 micrometer, optionally equal to or less than 7.0 micrometer, optionally equal to or less than 6.5 micrometer and optionally equal to or less than 6.0 micrometer, and the bacterial particles optionally have a cell viability, optionally viable cell count, of equal to or more than 2.00·10¹⁰ CFU/gram, optionally of equal to or more than 2.10·10¹⁰ CFU/gram, optionally of equal to or more than 1.50·10¹¹ CFU/gram and optionally of equal to or more than 2.00·10¹¹ CFU/gram.

32. A food or beverage product or a medicament for animal and/or human consumption comprising bacterial particles or a composition according to claim 31.

33. A method of producing a food or beverage product, the method comprising adding the bacterial particles or compositions of claim 31 to a process of making the food or beverage product.

34. A method of making a probiotic composition, the method comprising adding the bacterial particles or compositions of claim 31 to a process of making the probiotic composition.

35. A method of treating or preventing a disease or condition related to an animal or human gastrointestinal tract, in an animal or human in need thereof, the method comprising providing a therapeutically effective amount of the bacterial particles or compositions of claim 31 to the animal or human.
